(19) United States
(12) Patent Application Publication
(10) Pub. No.: US 2003/0104084 A1
(43) Pub. Date: Jun. 5, 2003

(54) Process for obtaining solid soy isoflavone -containing products

(76) Inventors: Ofir Ramot, Kibbutz Hatzor (IL); Trevor Meredith, Kibbutz Hatzor (IL)

Correspondence Address: NEEDLE & ROSENBERG P C, 127 PEACHTREE STREET N E, ATLANTA, GA, 30303-1811, US

(21) Appl. No.: 10/221,901

(22) PCT Filed: Oct. 14, 2001
(86) PCT No.: PCT/IL01/00948

(30) Foreign Application Priority Data
Jan. 17, 2001 (IL) 140953

(51) Int. Cl. A61K 35/78

(52) U.S. Cl. 424/757

(57) ABSTRACT

A process for obtaining a solid soy isoflavone- and protein-containing isolate in which isoflavones constitute at least 7% of the isolate and are present in predominantly glycoside form, (genistin+genistein) constitute at least 50% of the isoflavones, the weight ratio (genistin+genistein):(daidzin+daidzein) is at least 1.5:1 and the bulk density is at least 0.57 g/ml. The process includes the sequential steps of (a) heating a soy soluble extract (SSE), made by aqueous alcohol extraction of defatted soy solids and evaporation of the alcohol, at acid pH and at a temperature within the range 70°-90° C. to cause sedimentation of an insoluble fraction containing saponin, trypsin inhibitor and isoflavone compounds; (b) cooling the mixture from step (a) to a temperature of not lower than 30° C. and separating the insoluble fraction from supernatant liquid in the mixture of step (a) at a temperature of not lower than 30° C.; and (c) drying the separated insoluble fraction in a drying system adapted to produce a bulk density of at least 0.57 g/ml in the solid isolate.

### FIELD AND BACKGROUND OF THE INVENTION

The present invention relates to a process for obtaining solid soy isoflavone-containing products, and to products obtainable by the process.

Isoflavone constituents of soybeans are known to have desirable therapeutic and dietary applications, see e.g. U.S. Pat. No. 5,506,211 (Barnes et al.), U.S. Pat. No. 5,654,011 (Jackson et al.), U.S. Pat. No. 5,932,221 (Day), U.S. Pat. No. 5,952,374 (Clarkson, Jr., et al.), U.S. Pat. No. 6,001,368 (Jenks) and U.S. Pat. No. 6,150,399 (Patel et al.), and patent and literature references mentioned in these patents. Isoflavones have been recovered from soy molasses (see e.g. U.S. Pat. No. 6,053,553 to Waggle), which is an aqueous alcohol extract made by Chajuss from soybeans (IL 19168) and which has been recognized as having desirable therapeutic properties (IL 119107 and U.S. Pat. No. 5,871,743, to Chajuss).

The entire contents of the above-mentioned US and IL patents, and of the patent and literature references mentioned therein, are deemed to be incorporated by reference in the present patent application.

Among isoflavone constituents of soybeans, genistin and genistein have been the subject of numerous scientific papers in relation to, for example, a broad spectrum anti-cancer activity, estrogenic activity, prevention of osteoporosis, inhibition of platelet formation and prevention of atherosclerosis. Consequently, soy products containing a relatively high proportion of genistin and genistein are in much commercial demand.

One of the objects of the present invention is to provide an economically viable process for producing a soy product containing a relatively high proportion of genistin and genistein. Other objects of the invention will appear from the description which follows.

### SUMMARY OF THE INVENTION

The present invention accordingly provides a process for obtaining a solid soy isoflavone- and protein-containing isolate in which: isoflavones constitute at least 7% of said isolate, and are present in predominantly glycoside form, (genistin+genistein) constitute at least 50% of the isoflavones, the weight ratio (genistin+genistein):(daidzin+daidzein) is at least 1.5:1, and the bulk density is at least 0.57 g/ml.

This process includes the following sequential steps:

(a) heating a soy soluble extract (SSE), made by aqueous alcohol extraction of defatted soy solids and evaporation of the alcohol, at acid pH and at a temperature within the range 70°-90° C. to cause sedimentation of an insoluble fraction containing saponin, trypsin inhibitor and isoflavone compounds;

(b) cooling the mixture from step (a) to a temperature of not lower than 30° C. and separating said insoluble fraction from supernatant liquid in said mixture at said temperature; and

(c) drying said separated insoluble fraction in a drying system adapted to produce said bulk density in said solid isolate.

This process may be further characterized by at least one, and possibly all, of the following features:

(i) the SSE has a solid content of about 40% to about 70%;

(ii) the acid pH is within the range of about 1.5 to about 3.0;

(iii) the temperature in step (b) lies within the range of from about 30° C. to about 40° C.;

(iv) the separating is effected by means of centrifugation;

(v) the insoluble fraction is subjected to washing with water, prior to the drying step;

(vi) the drying system is effective to produce the solid isolate having a moisture level of not more than 8%;

(vii) the isolate contains not less than 20% saponins and 20 TIU/mg trypsin inhibitor;

(viii) the isolate is in the form of a powder;

(ix) prior to or after step (c), the insoluble fraction is subjected to treatment with aqueous alkali under conditions such that a major part of esterified isoflavone glycosides are converted to non-esterified glycosides;

(x) prior to or after step (c), the insoluble fraction isolate is subjected to chromatographic enrichment in order to increase the proportion of isoflavones in the product, such that it preferably contains at least 40% isoflavones.

The present process can however be adapted, if desired, to obtain a product which contains 60% and upwards, of isoflavones.

In a particular embodiment, the supernatant liquid from step (b) is subjected to a chromatographic separation in order to obtain therefrom an eluate which is subjected to a drying step to give a solid soy isoflavone- and protein-containing product in which; isoflavones constitute at least 40% of said product, and are present in predominantly glycoside form, (genistin+genistein) constitute at least 50% of the isoflavones, the weight ratio (genistin+genistein):(daidzin+daidzein) is at least 1.5:1, and the bulk density is at least 0.57 g/ml, the drying step being effected in a drying system adapted to produce this bulk density in the solid product.

This embodiment of the process may be further characterized by at least one, and possibly all, of the following features:

(i) the SSE has a solid content of about 40% to about 70%;

(ii) the acid pH is within the range of about 1.5 to about 3.0;

(iii) the temperature in step (b) lies within the range of from about 30° C. to about 40° C.;

(iv) the separating is effected by means of centrifugation;

(v′) the chromatographic separation includes a step of washing out with water, water-soluble material prior to obtaining the eluate;

(vi′) the drying system is effective to produce the solid product having a moisture level of not more than 8%;

(vii′) the chromatographic separation includes a step of washing out with water containing a minor amount of ethanol, a daidzin-rich fraction;

(viii′) the solid product is in the form of a powder;

(ix′) prior to the chromatographic separation, the supernatant liquid is subjected to treatment with aqueous alkali under conditions such that a major part of esterified isoflavone glycosides are converted to non-esterified glycosides.

The present invention extends to a substance which is a solid soy isoflavone- and protein-containing isolate obtained according to the process of the invention, or a solid soy isoflavone- and protein-containing product obtained according the embodiment where this product is obtained from the supernatant, and especially such substance in which (genistin+genistein) constitute at least about 55% of the isoflavones; and which has a bulk density of at least 0.57 g/ml, e.g. within the range of about 0.75 to about 0.85 g/ml.

The present invention additionally includes a solid powdered soy isoflavone- and protein-containing isolate which comprises at least daizdin, genistin and glycitin, and optionally at least one member selected from the group consisting of daizdein, genistein and glyciein, and the acetates and malonates of daizdin, genistin and glycitin, having a bulk density at least 0.57, wherein isoflavones constitute at least 7% of said isolate, in which genistin and genistein together constitute at least 55% (preferably at least 60%, more preferably at least 65%) of the isoflavone content of said isolate, and the weight ratio (genistin+genistein):(daidzin+daidzein) is at least 1.5:1, preferably at least 2:1. For specialized purposes it is possible to make a product in which genistin and genistein together constitute a higher proportion (e.g. 72% or more) of the isoflavone content of said isolate, and/or the weight ratio (genistin+genistein):(daidzin+daidzein) is at least 2.5:1.

While the SSE starting material usually contains about 20-25% oligosaccharides, this percentage is diminished in the present powdered isolate. Thus, in the present 7% isoflavones product there will usually be about 5-6% oligosaccharides, while the present product which contains at least 40% isoflavones will usually contain essentially no oligosaccharides.

The present invention further provides a therapeutic or dietary composition which comprises at least one soy isolate made by the process according to the invention, or as defined in the preceding paragraph, together with at least one carrier, diluent or adjuvant.

### Definitions

In the present specification and claims, the following definitions apply:

The phrase “isoflavones . . . are present in predominantly glycoside form” is intended to convey that at least 90%, preferably at least 95%, of the isoflavones present, including esterified derivatives, are in glycoside form.

Percentages mean percentages by weight, and “parts” means parts by weight, unless indicated otherwise.

### DETAILED DESCRIPTION OF THE INVENTION

The raw material to be used in the process of the present invention is a Soluble Soy Extract (SSE) obtained by an aqueous alcohol extraction of defatted soy solids, such as defatted flake produced from sound, cleaned soybeans. To ensure that all of the alcohol used for extraction has been removed from the SSE by the evaporation process, the solid content of the soluble soy extract should be preferably more than 40% but not higher than 70%. Concentrations lower than 40% and above 70% may affect the efficiency of the process.

The SSE has preferably a solid content in the range of 40% to 55%. Solid levels outside this range will affect the yield of saponins, isoflavones trypsin inhibitors as well as the concentration of these compounds in the obtained product.

In step (a), the SSE is heated at acid pH at a temperature within the range 70°-90° C. This may be done, e.g., by heating to the desired temperature and immediately contacting the SSE with a suitable acid in order to achieve an acid pH of e.g. about 1.5 to about 3. It is most preferred that the pH be adjusted to a pH of from 2.0 to 2.5. The preferred suitable acid may be e.g. sulfuric acid, nitric acid, phosphoric acid or hydrochloric acid, but other suitable acids, bearing in mind that the product is intended for human consumption, may alternatively be used.

Following the heating and pH change, the SSE must be cooled in step (b) without undue delay, preferably substantially immediately, and held for a period of time which is preferably such as to allow maximum precipitation of the insoluble components. The preferred temperature to which the product is cooled should range from about 30° C. to about 40° C. Temperatures below 30° C. may produce difficulties in separation of the sediment at the optimum solid concentration while those above 40° C. will allow only partial precipitation of the insoluble fractions. Temperatures outside of this range will also affect the ratio of the genistin to daidzin forms of the concentrated soy extract obtained as a result of the process. Optimum precipitation of the insoluble fraction will occur if the cooled liquid is allowed to stand for e.g. about 5 hours to about 10 hours at the preferred temperature range of 30° C. to 40° C., before the sediment is separated.

Also as part of step (b), the insoluble fraction is then separated from the remaining soluble fraction (supernatant) by any suitable means such as centrifugation. Preferably, the use of a centrifuge capable of applying a G force of above 4000 for a period of about 30 seconds at the designated holding temperature of 30° C. to 40° C. will cause separation of almost the entire sediment fraction from the supernatant.

In step (c) of the process, various methods for drying the insoluble fraction may be employed, for instance freeze-drying, spray drying, vacuum tray drying, or preferably using an air mill as the drying system, provided that the intended bulk density of at least 0.57 g/ml is achieved.

In an air mill drying system, the wet sediment is pumped into the lower section of the air mill and mixed with the hot air entering from the base of the air mill. Due to the high centrifugal forces applied, the wet material is spread on to the hot walls of the air mill and drying begins as a result of the upwards flow of the hot air over the wet sediment. At the same time, the now hot sediment also moves up the outer heated walls of the air mill. The dried material then flakes from the wall and further drying and grinding takes place in the upper sections of the mill. The finely ground material exiting from the air mill is then carried by the hot air through a pneumatic drying system where addition drying occurs. The resulting product may then be collected in a suitable separating device such as bag filter or high efficiency cyclone. The temperature of the sediment in the drying stage should preferably be restricted to less than 100° C. to avoid degradation of various heat sensitive compounds therein. The resulting dry solid isolate will preferably have a bulk density of not less than 0.75 g/ml. The choice of the preferred drying system should be such that the dried solid isolate will not have a moisture level of more than 8%.

The product of the present process preferably contains not less than 20% saponins, 20 Trypsin Inhibitor Units/mg. of product and at least 7%, e.g. 7-11%, of isoflavones. In the product of the process, (genistin+genistein) constitute at least 50%, preferably at least 55% of the isoflavones, and the weight ratio (genistin+genistein):(daidzin+daidzein) is at least 1.5:1, e.g. in the range of from 1.5:1 to 2.0:1, dependant on the quality and type of soybeans used.

In a further optional feature of the process, number (v) above, water may be added to the separated wet sediment (insoluble fraction), e.g. at a ratio of from about 10:1 to 1:1, preferably in a ratio of from about 3:1 to about 2:1. The resulting mixture is mixed for a period of e.g. 5 to 10 minutes at a temperature of e.g. from 30° C. to 40° C., followed by separation of the washed sediment by centrifugation as described in the initial separation step. The washing step aids in the removal of undesirable components from the insoluble sediment fraction. The resulting washed sediment is then dried as described previously. The dried product obtained after this optional water washing step is applied, preferably contains, e.g., 8-14% isoflavones.

In a further embodiment of the process of the present invention, see (x) above, the insoluble fraction isolate may be subjected to chromatographic enrichment in order to increase the proportion of isoflavones in the product. In still another embodiment of the process, supernatant liquid from step (b) may be subjected to a chromatographic separation in order to obtain therefrom an eluate which is subjected to a drying step to give a solid soy isoflavone- and protein-containing product as defined herein, in which higher concentrations of the isoflavone fractions may be achieved.

A preferred type of chromatographic absorbent for carrying out the embodiments outlined in the preceding paragraph would be e.g. a reverse phase type of resin. The isolate prior to or after step (c) may be dissolved in water at an isolate:water ratio of from 1:1 to 1:100 and preferably in a 1:30 ratio; alternatively, the supernatant from step (b) may be used directly for a chromatographic separation. The pH of the aqueous isolate solution, or the supernatant, may be adjusted to a pH in the range of from 10-10.5 and held at this pH for about 60 minutes with continuous mixing. The pH is then again adjusted to a pH from 6 to 10.5 but preferably from 7.0 to 8.0. The temperature is preferably held in a range of from 20°-60° C. Any sediment remaining in the resulting solution should then be separated, e.g. by filtration or centrifugation and the recovered liquid phase (solution) is then passed through a column containing the designated absorbent.

The absorbent in the column is then washed with water to remove any remaining unbound and undesired impurities from the solution. Removal and collection of the various desired fractions absorbed on the absorbent is performed by passing a suitable aqueous alcohol through the column. By using different ratios of alcohol to water, and by selecting the time frame for collection of the eluent, a substantial part of the isoflavones attached to the absorbent are released and can be collected to obtain a purified form of the desired product. The preferred aqueous alcohols include aqueous methanol, aqueous ethanol, aqueous isopropanol and aqueous propanol. The alcohol to water ratio should be from 20:80 to 80:20 depending on the type of alcohol used and purity of the liquid phase feed. The preferred alcohol in the present invention is aqueous ethanol.

Various methods, as described above, for drying the purified solid isolate may be employed, and the previous remarks, e.g. as to a desirable drying temperature of less than 100° C., and the desirability of the product having a moisture level of no more than 8%, are applicable. Preferably, the product obtained will contain at least about 40% isoflavones.

In a further embodiment of the present process, see (ix) and (ix′) above, the insoluble fraction prior to or after step (c), or the supernatant liquid prior to chromatographic separation, may be subjected to treatment with aqueous alkali under conditions such that a major part of the esterified isoflavone glycosides are converted to non-esterified glycosides. For this purpose, the insoluble fraction may be mixed with water at about a 1:3 to 1:100 ratio, but preferably at about a 1:30 ratio, and the obtained mixture, or the supernatant from step (b), may be treated with a suitable alkali to achieve pH from 7 to 10.5. The pH should preferably be in a range from 9.5 to 10.5. Preferably one of the following alkalies should be used: sodium hydroxide, potassium hydroxide or ammonium hydroxide. The high pH solution is then held at a temperature of from e.g. 20° C. to 100° C. for a period of time of e.g. up to 4 hours to convert esterified isoflavone glycosides to non-esterified glycosides. By holding both the pH and the temperature to the above-mentioned conditions, a major part, and preferably all or almost all, of the esterified isoflavone glycosides will be converted to non-esterified glycosides in less than 30 minutes.

Subsequent to the operation described in the preceding paragraph, the resulting glycoside solution may subjected to chromatography as described above.

The invention will now be illustrated by the following non-limiting examples, in which the analytical method used to determine the isoflavone percentages was as follows. The Isoflavone fraction in the sample was extracted by adding 0.05 gram of the sample to be tested to 25 ml of 80% HPLC grade methanol in water w/w. The sample was mixed by using a vortex stirrer and then placed in a shaking water-bath for two hours at 30° C. An aliquot was then filtered through a 0.45 um PTFE filter, and analyzed by HPLC. A VARIAN LC STAR Detector with a SPARK HOLLAND Model 9300 auto sampler, and a VARIAN STAR Model 9012 pump. A VARIAN STAR WORKSTATION VER. 5.0 data processing software was used to determine the isoflavone levels. A Hypersil ODS-2 C-18 column (5 um, 25 cm×4.6 mm) was employed for chromatographic separations and maintained at 35° C. with a column heater. Linear gradients were used with solution A composed of 0.1% acetic acid in water and solution B composed of 0.1% acetic acid in acetonitrile. A 20 μL sample is injected with initial conditions set at 14% B. The system was maintained at-these conditions for 13 min. B was then increased to 29% in 33 min and then to 31% until 44 minutes. This was then followed by a wash period until 54 minutes with B reaching 90%. An equilibration period of 20 minutes at the initial conditions was used between each injection. The flow rate was maintained at 1.5 ml during the complete cycle period. The UV absorbance was monitored at 254 nm. Pure samples of genistein, genistin, daidzein, daidzin, glycitin and glycitein were used to determine response values. Response values for the acetyl and malonyl forms were determined by adjusting the standard curve of the corresponding isomer based on molecular weight values. Results are expressed in the glycoside and aglycone form on an as is moisture basis. This analytical protocol is based on a method published by Pat Murphy of Iowa State University published in the J. Agric. Food Chem. Vol. 42 No 8 1994. pp. 1666-1673.

### EXAMPLE 1

A soy soluble extract (SSE) was at first heated to a temperature of about 80° C. followed by a pH adjustment with sulfuric acid to 2.4, immediately cooled to about 40° C., held for about 5 hours at this temperature, and the resulting sediment was separated from the supernatant by centrifugation. The centrifuge used applied a G force of 4000 g with a liquid retention time of about 30 seconds. Water (3 parts) was then added to the wet sediment. The water and sediment were thoroughly mixed for a period of 10 minutes then the washed sediment was separated from the liquid fraction by centrifugation as just described. The wet washed sediment was pumped into an air mill operating at an exit air temperature of 95° C. The dried isolate was collected in a bag filter with any coarse material being screened and fed back into the air mill for further grinding.

Samples, dried before analysis, were taken of the SSE and the wet sediment after the first centrifugal separation stage. An additional sample of the supernatant liquid was taken to determine the percentage of dry solid it contained. A sample of the washed and dried soy isolate from the air mill was also taken for analysis. The isoflavone percentages, moisture and bulk density of all samples was determined. Results are shown in Table 1.

TABLE 1

| | Soy | Unwashed | | Washed | |
|---|---|---|---|---|---|
| | Soluble | Concentrated | | Concentrated | |
| | Extract | Soy Isolate | | Soy Isolate | |
| Protein % | 7% | 17% | | 19% | |
| Moisture % | 47% | | | 6% | |
| | Dry solids | | | | |
| Bulk Density g/ml | — | 0.80 | g/ml | 0.80 | g/ml |
| Isoflavone Forms | % Isoflavone | % Isoflavone | | % Isoflavone | |
| 1. Daidzin | 0.25 | 1.09 | | 1.59 | |
| 2. Genistin | 0.30 | 1.97 | | 2.61 | |
| 3. Glycitin | 0.04 | 0.23 | | 0.34 | |
| 4. Malonyl Daidzin | 0.34 | 1.93 | | 2.56 | |
| 5. Malonyl Genistin | 0.43 | 3.42 | | 4.39 | |
| 6. Malonyl Glycitin | 0.06 | 0.33 | | 0.46 | |
| 7. Acetyl Daidzin | 0.02 | 0.16 | | 0.22 | |
| 8. Acetyl Genistin | 0.02 | 0.18 | | 0.24 | |
| 9. Acetyl Glycitin | 0.02 | 0.16 | | 0.24 | |
| 10. Daidzein | 0.01 | 0.09 | | 0.12 | |
| 11. Genistein | 0.01 | 0.11 | | 0.14 | |
| 12. Glycitein | 0.00 | 0.00 | | 0.02 | |
| Total Isoflavones | 1.50% | 9.67% | | 12.95% | |
| Genistin & Genistein | 50% | 59% | | 57% | |
| in Isoflavones | | | | | |
| Ratio of Genistin | 1.20:1 | 1.74:1 | | 1.64:1 | |
| forms to Daidzin | | | | | |
| forms | | | | | |

In this and subsequent tables, “genistin in isoflavones” and “genistin forms” include genistin, malonyl genistin and acetyl genistin, and the same applies to the daidzin and glycitin forms. All isoflavone results are expressed in glycoside and aglycone forms. The esterified glycosides have been mathematically adjusted to non-esterified glycosides using their molecular weight values.

### EXAMPLE 2

Washed and dried soy isolate (1 part) obtained similarly to Example 1 was dissolved in water (30 parts), the pH was adjusted to about 10 with 40% aq. NaOH, the resulting solution was heated to 100° C., cooled to about 30° C. while continually monitoring to maintain the pH in the range of about 9.5-10.0. The solution was then adjusted to pH about 7.5 and filtered through a 5 micron filter and the filtrate loaded onto a reverse phase resin column until saturated with isoflavones. After washing the column to remove residual sugars and other impurities, the isoflavone fractions were eluted with 30:70 ethanol-water. The eluate was collected, the ethanol fraction was evaporated and a liquid containing substantially de-esterified soy isolate was obtained, which was then freeze-dried in vacuum, to give a solid de-esterified soy isolate. Both starting material and the product were analyzed to determine isoflavone concentration and profile. The results are shown in Table 2.

TABLE 2

| | | De-esterified |
|---|---|---|
| | Starting material | product |
| Protein % | | 8% |
| Isoflavone Isomer Forms | % Isoflavone | % isoflavone |
| 1. Daidzin | 1.26 | 14.03 |
| 2. Genistin | 2.16 | 27.70 |
| 3. Glycitin | 0.31 | 3.70 |
| 4. Malonyl Daidzin | 2.11 | 0.50 |
| 5. Malonyl Genistin | 3.71 | 0.08 |
| 6. Malonyl Glycitin | 0.47 | 0.01 |
| 7. Acetyl Daidzin | 0.22 | 0.05 |
| 8. Acetyl Genistin | 0.28 | 0.00 |
| 9. Acetyl Glycitin | 0.21 | 0.00 |
| 10. Daidzein | 0.12 | 0.01 |
| 11. Genistein | 0.14 | 0.00 |
| 12. Glycitein | 0.03 | 0.00 |
| Total Isoflavones | 11.02% | 46.08% |
| Genistin & Genistein in | 57% | 60% |
| Isoflavones | | |
| Genistin forms:Daidzin forms | 1.69:1 | 1.88:1 |

### EXAMPLE 3

A supernatant obtained similarly to Example 1 was adjusted to pH about 10 at 30° C. by the use of a 40% solution of sodium hydroxide. The resulting solution was held at this pH and temperature with continuous monitoring for a period of 10 minutes. The resulting solution was then immediately adjusted to a pH of 8.0 and the solution centrifuged. The supernatant was immediately loaded onto a reverse phase resin column until saturated with isoflavones. After washing the column to remove residual sugars and other undesired impurities, a daidzin rich fraction of isoflavone was eluted from the column using 25:75 ethanol-water. The column was then eluted using 80:20 ethanol-water; the eluate was collected, the ethanol removed by evaporation and a liquid containing a de-esterified genistin rich isoflavone fraction was obtained. The liquid was freeze-dried in vacuum freeze drier to obtain solid de-esterified genistin rich isoflavone fraction. The supernatant used as the raw material and the resulting product were both analyzed to determine the isoflavone concentration and profile. The results are shown in Table 3.

TABLE 3

| | Starting material | De-esterified |
|---|---|---|
| | (Supernatant) | product |
| Protein % | 6% | 10% |
| Isoflavone Isomer Forms | % Isoflavone | % Isoflavone |
| 1. Daidzin | 0.23 | 14.88 |
| 2. Genistin | 0.22 | 29.07 |
| 3. Glycitin | 0.06 | 3.20 |
| 4. Malonyl Daidzin | 0.25 | 1.09 |
| 5. Malonyl Genistin | 0.30 | 10.03 |
| 6. Malonyl Glycitin | 0.04 | 0.38 |
| 7. Acetyl Daidzin | 0.02 | 0.73 |
| 8. Acetyl Genistin | 0.01 | 0.32 |
| 9. Acetyl Glycitin | 0.01 | 0.71 |
| 10. Daidzein | 0.01 | 0.23 |
| 11. Genistein | 0.01 | 0.05 |
| 12. Glycitein | 0.00 | 0.09 |
| Total Isoflavones | 1.16% | 60.76% |
| Genistin & Genistein in Isoflavones | 47% | 65% |
| Genistin forms:Daidzin forms | 0.94:1 | 2.32:1 |

While particular embodiments of the invention have been particularly described hereinabove, it will be appreciated that the present invention is not limited thereto, since as will be readily apparent to skilled persons, many modifications or variations can be made. Such modifications or variations which have not been detailed herein are deemed to be obvious equivalents of the present invention.

1. A process for obtaining a solid soy isoflavone- and protein-containing isolate in which:
isoflavones constitute at least 7% of said isolate, and are present in predominantly glycoside form,
(genistin+genistein) constitute at least 50% of the isoflavones, the weight ratio (genistin+genistein):(daidzin+daidzein) is at least 1.5:1, and
the bulk density is at least 0.57 g/ml,
which process includes the following sequential steps:
(a) heating a soy soluble extract (SSE), made by aqueous alcohol extraction of defatted soy solids and evaporation of the alcohol, at acid pH and at a temperature within the range 70°-90° C. to cause sedimentation of an insoluble fraction containing saponin, trypsin inhibitor and isoflavone compounds;
(b) cooling the mixture from step (a) to a temperature of not lower than 30° C. and separating said insoluble fraction from supernatant liquid in said mixture at said temperature; and
(c) drying said separated insoluble fraction in a drying system adapted to produce said bulk density in said solid isolate.

2. A process according to claim 1, which is further characterized by at least one of the following features:
(i) said SSE has a solid content of about 40% to about 70%;
(ii) said acid pH is within the range of about 1.5 to about 3.0;
(iii) said temperature in step (b) lies within the range of from about 30° C. to about 40° C.;
(iv) said separating is effected by means of centrifugation;
(v) said insoluble fraction is subjected to washing with water, prior to said drying step;
(vi) said drying system is effective to produce said solid isolate having a moisture level of not more than 8%;
(vii) said isolate contains not less than 20% saponins and 20 TIU/mg trypsin inhibitor;
(viii) said isolate is in the form of a powder;
(ix) prior to or after step (c), said insoluble fraction is subjected to treatment with aqueous alkali under conditions such that a major part of esterified isoflavone glycosides are converted to non-esterified glycosides;
(x) prior to or after step (c), said insoluble fraction isolate is subjected to chromatographic enrichment in order to increase the proportion of isoflavones in the product.

3. A process according to claim 2, wherein said chromatographic enrichment gives a product containing at least 40% isoflavones.

4. A process according to claim 1, wherein said supernatant liquid is subjected to a chromatographic separation in order to obtain therefrom an eluate which is subjected to a drying step to give a solid soy isoflavone- and protein-containing product in which:
isoflavones constitute at least 40% of said product, and are present in predominantly glycoside form,
(genistin+genistein) constitute at least 50% of the isoflavones, the weight ratio (genistin+genistein):(daidzin+daidzein) is at least 1.5:1, and the bulk density is at least 0.57 g/ml,
said drying step being effected in a drying system adapted to produce said bulk density in said solid product.

5. A process according to claim 4, which is further characterized by at least one of the following features:
(i) said SSE has a solid content of about 40% to about 70%;
(ii) said acid pH is within the range of about 1.5 to about 3.0;
(iii) said temperature in step (b) lies within the range of from about 30° C. to about 40° C.;
(iv) said separating is effected by means of centrifugation;
(v′) said chromatographic separation includes a step of washing out with water, water-soluble material prior to obtaining said eluate;
(vi′) said drying system is effective to produce said solid product having a moisture level of not more than 8%;
(vii′) said chromatographic separation includes a step of washing out with water containing a minor amount of ethanol, a daidzin-rich fraction;
(viii′) said solid product is in the form of a powder;
(ix′) prior to said chromatographic separation, said supernatant liquid is subjected to treatment with aqueous alkali under conditions such that a major part of esterified isoflavone glycosides are converted to non-esterified glycosides.

6. A process according to either claim 4 or claim 5, wherein said solid soy isoflavone-containing product contains at least 40% isoflavones.

7. A substance which is a solid soy isoflavone- and protein-containing isolate obtained according to the process of claim 1 or a solid soy isoflavone- and protein-containing product obtained according to claim 4.

8. A substance according to claim 7, in which (genistin+genistein) constitute at least about 55% of the isoflavones.

9. A solid powdered soy isoflavone- and protein-containing isolate which comprises at least daizdin, genistin and glycitin, and optionally at least one member selected from the group consisting of daizdein, genistein and glyciein, and the acetates and malonates of daizdin, genistin and glycitin, having a bulk density at least 0.57, wherein isoflavones constitute at least 7% of said isolate, genistin and genistein together constitute at least 55% of the isoflavone content of said isolate, and the weight ratio (genistin+genistein):(daidzin+daidzein) is at least 1.5:1.

10. A soy isolate according to claim 9, which possesses at least one of the following features:
isoflavones constitute at least 40% of said isolate;
the weight ratio (genistin+genistein):(daidzin+daidzein) is at least 2:1;
genistin and genistein together constitute at least 60% of the isoflavone content of said isolate.

11. A soy isolate according to claim 10, wherein genistin and genistein together constitute at least 65% of the isoflavone content of said isolate.

12. A therapeutic or dietary composition which comprises at least one soy isolate according to any one of claims 7 to 11, together with at least one carrier, diluent or adjuvant.
